(19) United States
(12) Patent Application Publication
(10) Pub. No.: US 2026/0368917 A1
(43) Pub. Date: Nov. 19, 2026

(54) INFORMATION PROCESSING DEVICE AND METHOD OF ESTIMATING NEONATE'S BODY WEIGHT

(71) Applicant: UNIVERSITY OF MIYAZAKI, Miyazaki (JP)

(72) Inventors: Kikuhito Kawasue, Miyazaki (JP); Masatoki Kaneko, Miyazaki (JP); Khin Dagon Win, Miyazaki (JP)

(73) Assignee: UNIVERSITY OF MIYAZAKI, Miyazaki (JP)

(21) Appl. No.: 19/133,589

(22) PCT Filed: Nov. 20, 2023
(86) PCT No.: PCT/JP2023/041637

§ 371 Date: May 28, 2025

(30) Foreign Application Priority Data
Dec. 1, 2022 (JP) 2022-192854

(51) Int. Cl. G06T 7/62; G06T 7/50

(52) U.S. Cl. CPC G06T 7/62; G06T 7/50; G06T 2207/30196

(57) ABSTRACT

A volume of a subject can be measured without touching the subject. An information processing device includes: an acquisition unit configured to acquire image information indicating a three-dimensional image obtained by capturing a subject lying on a flat surface from above; a recognition unit configured to recognize a subject image representing the subject among objects displayed in the three-dimensional image; and an estimation unit configured to estimate a volume of the subject including a lower portion missing in the subject image using the subject image, assuming that the lower portion of the subject is in contact with the flat surface. In addition, a neonate weight estimation method includes: capturing a three-dimensional image of a neonate; estimating, by a computer, a volume of the neonate from the three-dimensional image; and estimating, by the computer, a weight of the neonate from the estimated volume.

### TECHNICAL FIELD

The present invention relates to an information processing device and a neonate weight estimation method.

### BACKGROUND ART

Conventionally, a technology for acquiring various types of biological information from a subject (e.g., a neonate) lying on a flat surface (e.g. an upper surface of a mat) has been proposed. For example, Patent Document 1 discloses a technology for measuring a heart rate of a neonate in an incubator. For some neonates (particularly preterm neonates), touching them to acquire biological information may be invasive actions. According to the technology of Patent Document 1, the heart rate can be measured without touching the neonate, which is advantageous in that the number of invasive actions can be reduced.

### PRIOR ART DOCUMENTS

### Patent Documents

Patent Document 1: Japanese Patent Application Laid-Open No. 2022-78703

### SUMMARY OF THE INVENTION

### Problems to be Solved by the Invention

However, in the technology of Patent Document 1, it is necessary to touch the neonate to measure some types of biological information. For example, when a weight of the neonate is measured, it is necessary to touch the neonate. In consideration of the above circumstances, an object of the present invention is to enable measurement of biological information that requires touching a subject during the measurement in the prior art without touching the subject.

### Means for Solving the Problem

In order to solve the above problem, an information processing device according to the present invention includes: an acquisition unit configured to acquire image information indicating a three-dimensional image obtained by capturing a subject lying on a flat surface from above; a recognition unit configured to recognize a subject image representing the subject among objects displayed in the three-dimensional image; and an estimation unit configured to estimate a shape of the subject including a lower portion missing in the subject image using the subject image, assuming that the subject is in contact with the flat surface on a lower side, in which physical information of the subject is calculated using the estimated shape of the subject.

### Effects of the Invention

According to the present invention, biological information that requires touching a subject during measurement in the prior art can be measured without touching the subject,

### BRIEF DESCRIPTION OF THE DRAWINGS

FIG. 1 is a diagram for explaining each component of an information processing system.

FIG. 2 is a hardware configuration diagram of a computer.

FIG. 3 is a functional block diagram of an information processing system.

FIG. 4 is a diagram for explaining a specific example of a subject image,

FIG. 5 is another diagram for explaining a specific example of a subject image.

FIG. 6 is a diagram for explaining a configuration for generating a subject model,

FIG. 7 is a diagram for explaining a head size estimation process and the like.

FIG. 8 is a diagram for explaining a head model deformation process.

FIG. 9 is a diagram for explaining a body model deformation process.

FIG. 10 is a diagram for explaining a weight estimation process and a height estimation process.

FIG. 11 is a flowchart of image capturing processing.

FIG. 12 is a diagram for explaining a subject model according to a second embodiment.

FIG. 13 is a diagram for explaining elliptical approximation processing in a modification.

### BEST MODE FOR CARRYING OUT THE INVENTION

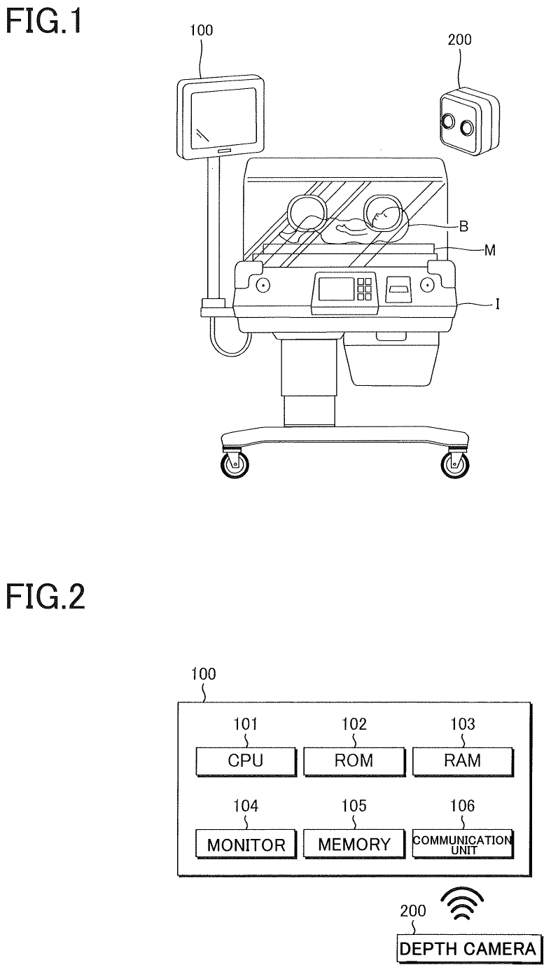

FIG. 1 is a diagram for explaining each component of an information processing system 1. The information processing system 1 of the present embodiment includes a computer 100 and a depth camera 200. The above components are communicably connected to each other. In the present embodiment, an incubator is employed as the computer 100. As illustrated in FIG. 1, a mat is provided inside the incubator. A neonate (hereinafter, “subject S”) lies on an upper surface (hereinafter, “flat surface A”) of the mat. The flat surface A is a plane having a substantially rectangular outer edge,

The depth camera 200 generates a three-dimensional image (distance image) including depth information indicating a distance to a subject. For example, a point cloud image captured using a light detection and ranging or laser imaging detection and ranging (LIDAR) technology is assumed as the three-dimensional image. In the present embodiment, a weight or height of the subject S is estimated by the computer 100 by capturing the subject S with the depth camera 200.

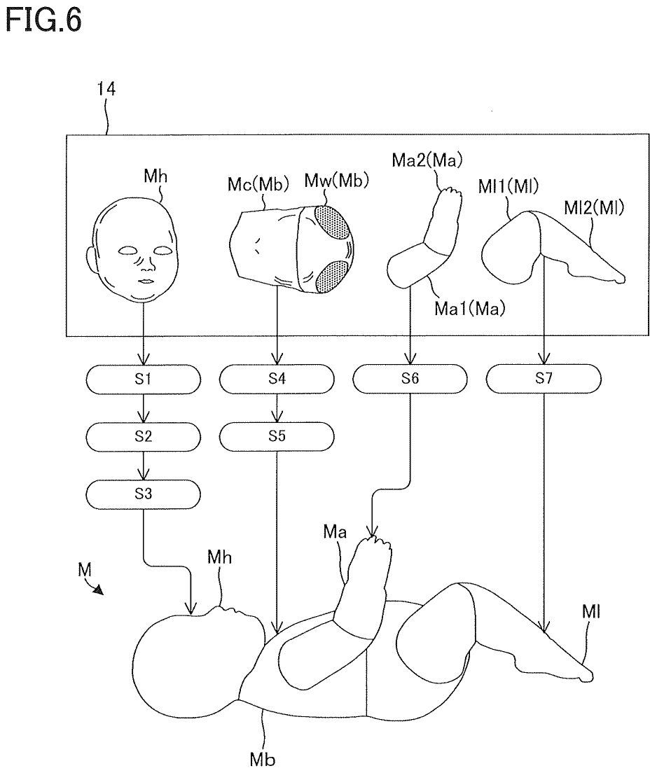

Specifically, when the subject S is captured by the depth camera 200, image information Dg of the three-dimensional image indicating the subject S is transmitted to the computer 100. The computer 100 generates a subject model M indicating a shape (size) of the captured subject S, using the image information Dg received from the depth camera 200 (see FIG. 6 to be described later). The volume of the subject model M is estimated as a volume of the captured subject S, and a weight of the subject S is estimated by multiplying an estimation result by a density. In addition, the height of the subject model M is estimated as a height of the captured subject S. The above configuration will be described in detail later.

In the present embodiment, the incubator is used as the computer 100, but the computer 100 may be configured separately from the incubator. In addition, the depth camera 200 may be configured to have the functions of the computer 100. In addition, the incubator may include the depth camera 200. Instead of the configuration in which the image information Dg is transmitted from the depth camera 200 to the computer 100 by wireless communication, the image information Dg may be transmitted by wired communication.

FIG. 2 is a hardware configuration diagram of the present embodiment. The computer 100 includes a central processing unit (CPU) 101, a read only memory (ROM) 102, a random access memory (RAM) 103, a monitor 104, a memory 105, and a communication unit 106.

The ROM 102 of the computer 100 stores various types of data including programs in a nonvolatile manner. The CPU 101 executes the programs to implement various functions (such as the estimation unit 13) to be described later. The RAM 103 temporarily stores, for example, various types of information to be referred to when the CPU 101 executes the programs. The monitor 104 displays various types of information. For example, the monitor 104 displays biological information of the subject S in the incubator.

The memory 105 stores various types of information in a nonvolatile manner. As the memory 105, for example, a flash memory can be employed. The memory 105 stores information necessary for estimating the weight and height of the subject S. Specifically, information indicating a head model Mh, a body model Mb, an arm model Ma, and a leg model Ml for generating the subject model M to be described later is stored in the memory 105. The communication unit 106 receives various types of information from an external device. For example, the image information Dg transmitted from the depth camera 200 is received by the communication unit 106.

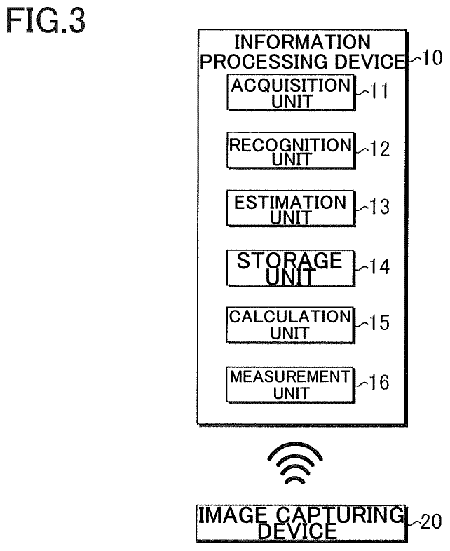

FIG. 3 is a functional block diagram of the information processing system 1 according to the present embodiment. As illustrated in FIG. 3, the information processing system 1 includes an information processing device 10 and an image capturing device 20. For example, the computer 100 described above functions as the information processing device 10 by executing the programs, and the depth camera 200 functions as the image capturing device 20. As described above, the image information Dg indicating a captured three-dimensional image G is transmitted from the image capturing device 20 to the information processing device 10.

As illustrated in FIG. 3, the information processing device 10 includes an acquisition unit 11, a recognition unit 12, an estimation unit 13, a storage unit 14, a calculation unit 15, and a measurement unit 16. The acquisition unit 11 acquires image information Dg indicating a three-dimensional image G of the subject S lying on the flat surface A captured from above.

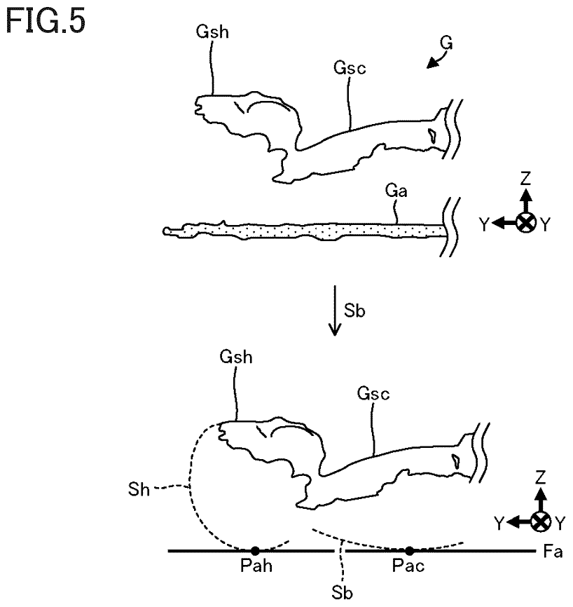

Specifically, the acquisition unit 11 acquires the image information Dg from the image capturing device 20. As will be described in detail later, the three-dimensional image G of the subject S lying on the flat surface A captured from above includes a flat surface image Ga representing the flat surface A in addition to a subject image Gs representing the subject S (see FIG. 5).

The recognition unit 12 recognizes the subject image Gs representing the subject S among objects displayed in the three-dimensional image. Specifically, the recognition unit 12 separately recognizes the subject image Gs representing the subject S and the flat surface image Ga representing the flat surface A. As a technology for recognizing each object in the three-dimensional image G, for example, a segmentation technology is suitably adopted. An artificial intelligence (AI) technology can be adopted for the segmentation. For example, each object in the three-dimensional image G is segmented using a trained fully convolutional network (FCN) (for example, a technology described in Japanese Patent Application Laid-Open No. 2022-29169 can be adopted).

The estimation unit 13 estimates a volume of the subject S using the subject image Gs. It is assumed that a subject image Gs representing the entire subject S (both an upper portion and a lower portion) is captured. In the above case, the volume of the subject S substantially matches the volume of the subject image Gs. Therefore, the volume of the subject image Gs representing the entire subject S can be estimated as the volume of the subject S.

Meanwhile, it is usual in reality that, in a case where the image of the subject S lying on the flat surface A is captured from above, only an image of the upper portion of the subject is captured, and an image of the lower portion (flat surface A side) is not captured (see FIG. 5 to be described later). Therefore, the lower portion of the subject S is missing in the subject image Gs. The volume of the subject image Gs cannot be used as an actual volume of the subject S.

In consideration of the above circumstances, the present embodiment adopts a configuration capable of estimating the volume of the subject S with high accuracy even from the subject image Gs in which the lower portion of the subject S is missing. Specifically, assuming that the subject S is in contact with the flat surface A on the lower side, the estimation unit 13 of the present embodiment estimates the volume of the subject S including the lower portion that is missing in the subject image Gs using the subject image Gs.

More specifically, the storage unit 14 stores model information indicating a shape of each model (such as the head model Mh) representing each portion (such as the head) of the subject S (see FIG. 6 to be described later). The model information of the present embodiment includes image information indicating the head model Mh, which is a model for the head of the subject S, image information indicating the body model Mb, which is a model for the body of the subject S, image information indicating the arm model Ma, which is a model for the arms of the subject S, and image information indicating the leg model Ml, which is a model for the legs of the subject S.

The estimation unit 13 deforms the shapes of the models (Mh, Mb, Ma, Ml), using the subject image Gs, so that the models approximates the shapes of the corresponding portions of the subject S. In addition, the estimation unit 13 generates a subject model M indicating an entire shape of the subject S using the deformed models. The estimation unit 13 estimates a volume of the subject model M as a volume of the subject S.

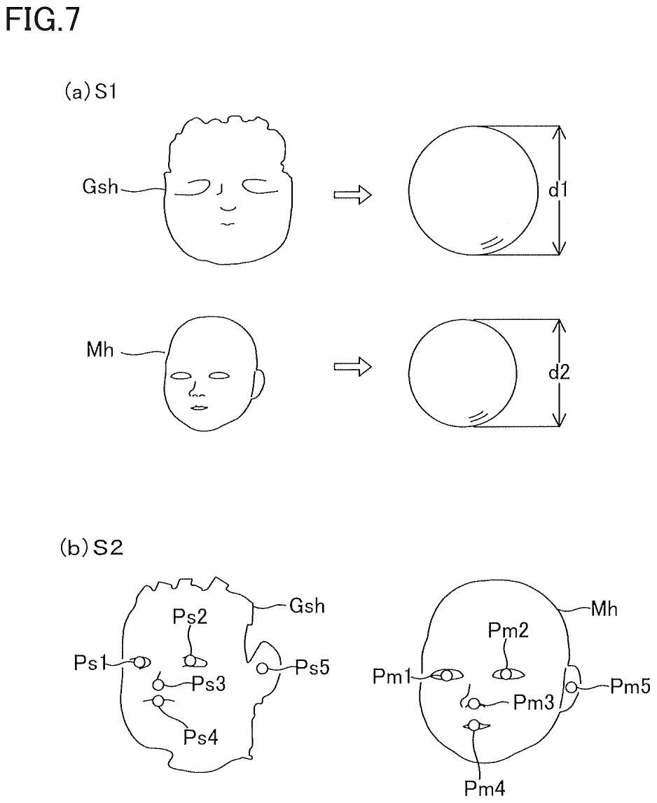

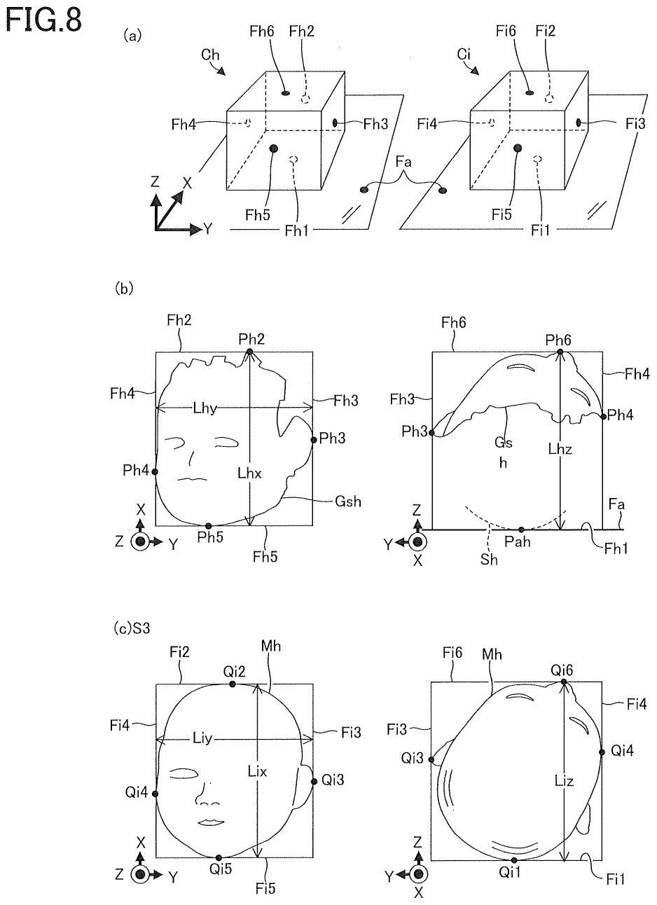

In addition, when deforming the shapes of the head model Mh and the body model Mb among the models, the estimation unit 13 deforms the shapes of the models assuming that the subject S is in contact with the flat surface A on the lower side. Specifically, the estimation unit 13 specifies a reference plane Fa passing through the flat surface image Ga (see FIG. 5). In addition, the estimation unit 13 deforms the models (Mh, Mb) based on a distance from the subject image Gs to the reference plane Fa (Lhz in FIG. 8(b)) (see FIGS. 7(a) and 7(b), FIGS. 8(a) to 8(c), and FIGS. 9(a) and 9(b)), and estimates the volume of the subject S using the deformed models. The above configuration will be described in detail later.

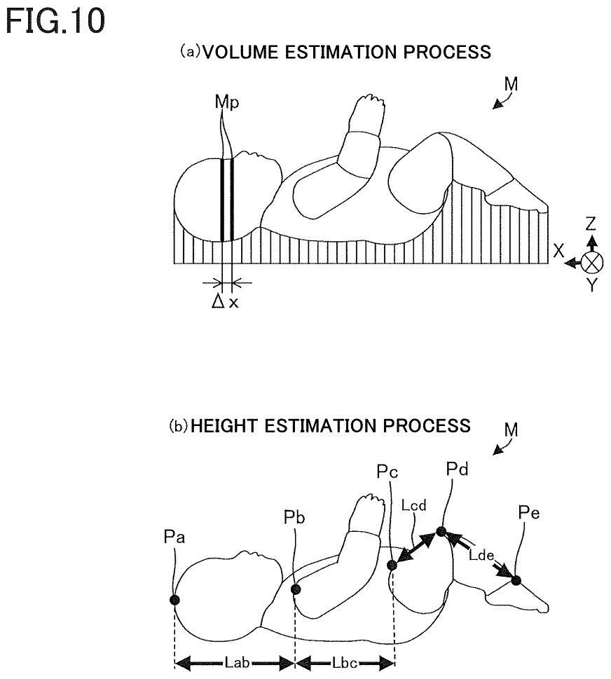

The calculation unit 15 calculates a weight of subject S using the volume of subject S estimated by the estimation unit 13. Specifically, the information processing device 10 stores an average density of the subject S (hereinafter, “average density D”) in advance. The calculation unit 15 calculates the weight W of the subject S by multiplying the volume V of the subject S estimated by the estimation unit 13 by the average density D (W=V×D). The measurement unit 16 measures a height of the subject S using the models deformed by the estimation unit 13. Specifically, the measurement unit 16 estimates a length of a predetermined portion in the subject model M as the height of the subject S (see FIG. 10).

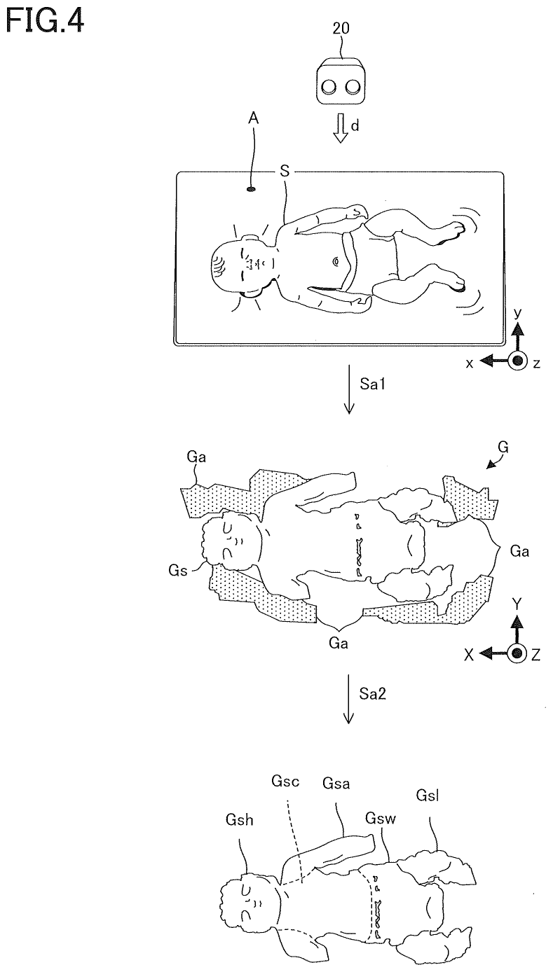

FIG. 4 is a diagram for explaining a specific example of a configuration until the subject image Gs is generated. In the present embodiment, a mat is provided inside the incubator such that the flat surface A is substantially horizontal to the horizontal direction. Hereinafter, the vertical direction may be referred to as “z-axis direction” for the sake of explanation. The length direction of the flat surface A may be referred to as “x-axis direction”, and the width direction of the flat surface A may be referred to as “y-axis direction”. In the above case, the x-y plane is substantially parallel to the horizontal direction. In the present embodiment, as illustrated in FIG. 4, it is assumed that the subject S lies on the flat surface A with the top of the head oriented in the x-axis direction.

In the present embodiment, as illustrated in an upper part of FIG. 4, a neonate lying on the flat surface A is captured as the subject S by the image capturing device 20 (depth camera 200). In the specific example of FIG. 4, it is assumed that the subject S is captured from above the subject S (in a direction indicated by arrow d). In the above case, the flat surface A is captured in addition to the subject S. When a three-dimensional image G is captured by the image capturing device 20, image information Dg indicating the three-dimensional image G is transmitted to the information processing device 10 (Sa1 in FIG. 4).

A simulated diagram of the three-dimensional image G captured by the image capturing device 20 is illustrated in a central portion of FIG. 4. Hereinafter, for the sake of explanation, a direction in a three-dimensional image space corresponding to the z-axis direction (vertical direction) may be referred to as “Z-axis direction”. Similarly, a direction corresponding to the x-axis direction may be referred to as “X-axis direction”, and a direction corresponding to the y-axis direction may be referred to as “Y-axis direction”, As illustrated in FIG. 4, the three-dimensional image G indicated by the image information Dg includes a subject image Gs and a flat surface image Ga. The subject image Gs represents the subject S, and the flat surface image Ga represents the flat surface A. In FIG. 4, for the sake of explanation, the subject image Gs and the flat surface image Ga are shown in different colors (the same applies to FIG. 5 to be described later).

The information processing device 10 recognizes each image in the three-dimensional image G, including the subject image Gs and the flat surface image Ga, using a segmentation technology (Sa2 in FIG. 4). For example, the subject image Gs and the flat surface image Ga are recognized as images indicating different objects.

In a lower portion of FIG. 4, the subject image Gs extracted from the three-dimensional image G is shown. As illustrated in FIG. 4, the subject image Gs includes a head image Gsh, a chest image Gsc, a waist image Gsw, an arm image Gsa, and a leg image Gsl. The head image Gsh represents the head of the subject S. Similarly, the chest image Gsc represents the chest from the neck to the navel in the body of the subject S, the waist image Gsw represents a portion from the navel to the legs in the body of the subject S, the arm image Gsa represents the arms of the subject S, and the leg image Gsl represents the legs of the subject S. Each of the above images is recognized as an image indicating a separate object using a segmentation technology.

FIG. 5 is a diagram for explaining the reference plane Fa. Although it will be described in detail later, the reference plane Fa is a plane passing through the flat surface A, and is used to estimate the volume of the subject S. FIG. 5 illustrates simulated diagrams of the three-dimensional image G (subject image Gs and flat surface image Ga). In the specific example of FIG. 5, it is assumed that the three-dimensional image G is viewed in the X-axis direction. Note that, in FIG. 5, the head image Gsh and the chest image Gsc are extracted from the subject image Gs and shown, and the other images (arm image Gsa, etc.) are omitted.

In the specific example of FIG. 5, similarly to the specific example of FIG. 4 described above, it is assumed that the subject S lying on the flat surface A is captured from above. In the above case, the subject image Gs includes an image representing an upper portion of the subject S, but does not include (lacks) a lower portion of the subject S. For example, in the specific example of FIG. 5, it is assumed that the subject S (neonate) lies face-up on the flat surface A. In the above case, the head image Gsh of the subject image Gs represents a face side (eyes, nose, and mouth) of the subject S, but does not include an occipital portion side of the subject S. In addition, the chest image Gsc of the subject image Gs represents a ventral side (chest and navel) of the subject S, but does not include a dorsal of the subject S.

The information processing device 10 of the present embodiment specifies the reference plane Fa from the flat surface image Ga (Sb in FIG. 5). Specifically, the flat surface image Ga is a point cloud image. In the present embodiment, the reference plane Fa is calculated by plane approximation using the least squares method. That is, a plane on which the sum of the squares of distances from the points constituting the flat surface image Ga is smallest is specified as the reference plane Fa.

FIG. 5 illustrates a conceptual diagram of the reference plane Fa. Note that, as described above, the flat surface image Ga is perpendicular to the Z-axis direction (vertical direction). Therefore, the reference plane Fa is also perpendicular to the Z-axis direction. The flat surface image Ga is parallel to the X-Y plane (horizontal direction). Therefore, the reference plane Fa is also parallel to the X-Y plane.

As described above, in a case where the subject S is captured from above, the lower portion of the subject image Gs is missing. In FIG. 5, a lower portion (Sh, Sb) of the subject S missing from the subject image Gs is indicated by a broken line. Specifically, in the subject image Gs, an occipital portion Sh missing from the head image Gsh and a dorsal portion Sb missing from the chest image Gsc are indicated by broken lines.

It is usual that, when the subject S (neonate) lies on the flat surface A, a lower end Pah of the occipital portion Sh of the subject S is in contact with the flat surface A. That is, although the lower end Pah of the occipital portion Sh of the subject S is missing from the subject image Gs, a point in the three-dimensional image space corresponding to the lower end Pah is located on the reference plane Fa, Similarly, it is usual that, when the subject S lies on the flat surface A, a lower end Pac of the dorsal portion Sb of the subject S is in contact with the flat surface A. That is, although the lower end Pac of the dorsal portion Sb of the subject S is missing from the subject image Gs, a point in the three-dimensional image space corresponding to the lower end Pac is located on the reference plane Fa.

FIG. 6 is a diagram for explaining a specific example of each process for generating the subject model M. As described above, the subject model M indicates the entire shape of the subject S. The information processing device 10 generates the subject model M from the captured subject image Gs, and estimates a volume of the subject model M as a volume of the subject S.

As illustrated in FIG. 6, model information indicating the head model Mh, the body model Mb, the arm model Ma, and the leg model Ml is stored in advance in the storage unit 14 of the information processing device 10. The body model Mb includes a chest model Mc and a waist model Mw.

The head model Mh (before deformed) stored in the storage unit 14 represents an average shape of the head of the subject S. Similarly, the body model Mb stored in the storage unit 14 represents an average shape of the body of the subject S, the waist model Mw stored in the storage unit 14 represents an average shape of the waist of the subject S, the arm model Ma stored in the storage unit 14 represents an average shape of the arms of the subject S, and the leg model Ml stored in the storage unit 14 represents an average shape of the legs of the subject S.

When acquiring the subject image Gs from the image capturing device 20, the information processing device 10 extracts the head image Gsh from the subject image Gs. Furthermore, the information processing device 10 executes a head size estimation process (S1 in FIG. 6) using the head image Gs and the head model Mh. In the head size estimation process, a rough shape (size) of the head model Mh is determined (see FIG. 7(a) to be described later). Furthermore, the information processing device 10 executes a head model rotation process (S2 in FIG. 6) after executing the head size estimation process. In the head model rotation process, the orientation of the head model Mh is rotated (adjusted) according to the orientation of the head image Gh (see FIG. 7(b)).

Thereafter, the information processing device 10 executes a head model deformation process (S3 in FIG. 6). As will be described in detail later, in the head model deformation process, assuming that the subject S is in contact with the flat surface A on the lower side, the shape of the head model Mh is deformed to approximate the shape of the head of the subject S (to the size of the head of the subject S) (see FIGS. 8(a) to 8(c) to be described later).

The information processing device 10 executes a body image rotation process (S4 in FIG. 6). In the body image rotation process, the orientation of the body model Mb and the orientation of the body image Gb are aligned. The body model Mb of the present embodiment is oriented in the X-axis direction in advance. In the body image rotation process, the body image Gb is rotated to be oriented in the X-axis direction. Specifically, the body image Gb is a point cloud image. The information processing device 10 specifies the orientation of the body image Gb using principal component analysis, and rotates the body image Gb to be parallel to the X-axis direction. As a configuration for adjusting the orientation of the point cloud image, for example, a configuration described in Japanese Patent Application Laid-Open No. 2014-44078 can be adopted.

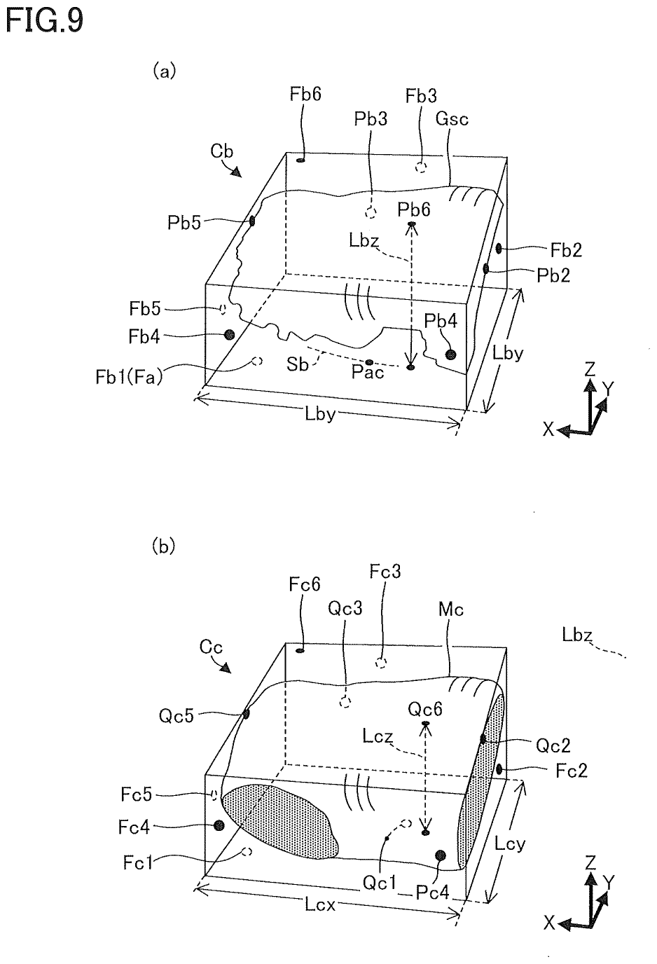

After executing the body image rotation process, the information processing device 10 executes a body model deformation process (S5 in FIG. 6). As will be described in detail later, in the body model deformation process, assuming that the subject S is in contact with the flat surface A on the lower side, the shape of the body model Mb is deformed to approximate the shape of the body of the subject S (see FIGS. 9(a) and 9(b) to be described later).

The information processing device 10 deforms the shape of the arm model Ma to approximate the shapes of the arms of the subject S by performing an arm model deformation process (S6 in FIG. 6). The above-described arm image Gsa includes an image representing a portion from the shoulder to the elbow of the subject S (hereinafter, “first arm image Gsa1”) and an image representing a portion from the elbow to the fingertip of the subject S (hereinafter, “second arm image Gsa2”). Each of the above images is distinguished using a segmentation technology. In addition, as illustrated in FIG. 6, the arm model Ma includes a first arm model Ma1 representing a portion from the shoulder to the elbow and a second arm model Ma2 representing a portion from the elbow to the fingertip.

In the arm model deformation process, the information processing device 10 deforms the first arm model Ma1 using the first arm image Gsa1. Similarly, the information processing device 10 deforms the second arm model Ma2 using the second arm image Gsa2 in the arm model deformation process. Note that the subject image Gs includes an arm image Ga representing the right arm of the subject S and an arm image Ga representing the left arm of the subject S. The arm model Ma also includes an arm model Ma representing the right arm and an arm model Ma representing the left arm of the subject S. In the arm model deformation process, the arm model Ma representing the right arm is deformed using the arm image Ga representing the right arm.

Similarly, the arm model Ma representing the left arm is deformed using the arm image Ga representing the left arm. Note that a specific example of the arm model deformation process will be described later as a modification.

The information processing device 10 deforms the shape of the leg model Ml to approximate the shapes of the legs of the subject S by performing a leg model deformation process (S7 in FIG. 6). The above-described leg image Gsl includes an image representing from the base of the leg to the knee of the subject S (hereinafter referred to as a “first leg image Gsl1”) and an image representing from the knee to the toe of the subject S (hereinafter referred to as a “second leg image Gsl2”). Each of the above images is distinguished using a segmentation technology. As illustrated in FIG. 6, the leg model Ml includes a first leg model Ml1 representing the thigh of the subject S and a second leg model Ml2 representing a portion from the knee to the toe of the subject S.

In the leg model deformation process, the information processing device 10 deforms the first leg model Ml1 using the first leg image Gsl1. Similarly, in the leg model deformation process, the information processing device 10 deforms the second leg model Ml2 using the second leg image Gsl2. Note that the subject image Gs includes a leg image Gsl representing the right leg of the subject S and a leg image Gsl representing the left leg of the subject S. The leg model Ml also includes a leg model Ml representing the right leg of the subject S and a leg model Ml representing the left leg of the subject S. In the leg model deformation process, the leg model Ml representing the right leg is deformed using the leg image Gsl representing the right leg. Similarly, the leg model Ml representing the left leg is deformed using the leg image Gsl representing the left leg. Note that a specific example of the leg model deformation process will be described later as a modification.

As understood from the above description, the head model Mh, the body model Mb (chest model Mc and waist model Mw), the arm model Ma, and the leg model Ml stored in the storage unit 14 are deformed according to the captured subject image Gs. The information processing device 10 generates a subject model M by appropriately combining the deformed models. The subject model M approximates the actual shape of the subject S. Hereinafter, the above-described configuration for deforming the head model Mh (see FIGS. 7(a) and 7(b) and FIGS. 8(a) to 8(c)) and the above-described configuration for deforming the body model Mb (see FIGS. 9(a) and 9(b)) will be described in detail.

FIG. 7(a) is a diagram for explaining the head size estimation process (S1 in FIG. 6) in detail. As described above, in the head size estimation process, an approximate size of the head model Mh is determined. Specifically, as illustrated in FIG. 7(a), the head image Gsh is spherically approximated by the least squares method using a point group constituting the head image Gsh. In addition, a diameter d1 of the spherically approximated head image Gsh is obtained. Similarly, the head model Mh before deformed is approximated to a sphere, and a diameter d2 of the spherically approximated head model Mh is obtained.

The information processing device 10 obtains a ratio Rd (Rd=d1/d2) between the diameter d1 of the spherically approximated head image Gsh and the diameter d2 of the spherically approximated head model Mh. Furthermore, the information processing device 10 changes the size of the head model Mh using the ratio Rd. Specifically, the head model Mh before deformed is multiplied by the ratio Rd. According to the above head size estimation process, the size of the head model Mh becomes close to the actual size of the head of the subject S. However, in the present embodiment, by executing the head model deformation process (see FIG. 8) in addition to the head size estimation process, the shape of the head model Mh is deformed to approximate the shape of the head of the subject S with high accuracy.

FIG. 7(b) is a diagram for explaining the head model rotation process (S2 in FIG. 6) in detail. As described above, in the head model rotation process, the orientation of the head model Mh is rotated (adjusted) according to the orientation of the head image Gh. Specifically, when the coordinates of an arbitrary point in the head image Gh (hereinafter, “subject point Ps”) are defined as (xi, yi, zi) and the coordinates of a point in the head model Mh corresponding to the subject point Ps (representing the same portion in the subject S) (hereinafter, “corresponding point Pm”) are defined as (xi′, yi′, zi′), the information processing device 10 obtains the following rotational movement transformation formula (r, t).

$$[ \begin{matrix}{x}_{i} \\ {y}_{i} \\ {z}_{i} \\ 1\end{matrix} ] = [ \begin{matrix}{r}_{11} & {r}_{12} & {r}_{13} & {t}_{1} \\ {r}_{21} & {r}_{22} & {r}_{23} & {t}_{2} \\ {r}_{31} & {r}_{32} & {r}_{33} & {t}_{3} \\ 0 & 0 & 0 & 1\end{matrix} ] [ \begin{matrix}{x}_{i}^{′} \\ {y}_{i}^{′} \\ {z}_{i}^{′} \\ 1\end{matrix} ] \qquad ( Mathematical formula 1 )$$

In order to obtain the rotational movement transformation formula (r, t), the information processing device 10 substitutes a combination of a specific subject point Ps and a corresponding point Pm corresponding to the subject point Ps. Specifically, a technology for detecting each feature point (pupil or the like) in a face from a face image has been conventionally known. As the above technology, for example, a technology described in Japanese Patent Application Laid-Open No. 2022-128652 can be adopted. The information processing device 10 detects a right eye Ps1, a left eye Ps2, a nose Ps3, a mouth Ps4, and a left ear Ps5 of the subject S in the head image Gs as subject points Ps. Similarly, the information processing device 10 detects a right eye Pm1, a left eye Pm2, a nose Pm3, a mouth Pm4, and a left ear Pm5 in the head model Mh as corresponding points Pm.

The information processing device 10 calculates elements r and elements t by substituting a plurality of sets of combinations of subject points Ps and corresponding points Pm, and obtains a rotational movement transformation formula. Specifically, the sum of the number of elements r to be calculated (9) and the number of elements t to be calculated (3) is 12. By substituting one set of a subject point Ps and a corresponding point Pm, three linear equations including elements r and elements t as coefficients are obtained. In the present embodiment, 15 linear equations are obtained by substituting the above-described five sets of combinations of subject points Ps and corresponding points Pm, and elements r and elements t are obtained using some of the linear equations.

The information processing device 10 moves the corresponding points Pm in the head model Mh to the subject points Ps in the subject image Gs using the rotational movement transformation formula. That is, the information processing device 10 rotates the orientation of the head model Mh to the orientation of the head image Gsh.

FIGS. 8(a) to 8(c) are diagrams for explaining the head model deformation process in detail. As described above, the shape of the head model Mh is deformed to approximate the shape of the head of the subject S with high accuracy by performing the head model deformation process.

FIG. 8(a) is a diagram for explaining a reference box Ch and an adjustment box Ci. As will be described in detail later, the reference box Ch is generated according to the size of the subject image Gs. In addition, the adjustment box Ci is generated according to the size of the head model Mh immediately before the head model deformation process is executed. In the head model deformation process, a ratio Rhi between the size of the reference box Ch and the size of the adjustment box Ci is obtained, and the head model Mh is deformed (enlarged or reduced) according to the ratio Rhi.

As illustrated in FIG. 8(a), the reference box Ch is a rectangular parallelepiped, and includes a bottom surface Fh1, side surfaces Fh2 to Ph5, and a top surface Fh6. In addition, as illustrated in FIG. 8(a), each surface Fh of the reference box Ch is parallel to one of the X-Y plane, the X-Z plane, and the Y-Z plane. Specifically, among the surfaces Fh1 to Fh6, the bottom surface Fh1 and the top surface Fh6 are parallel to the X-Y plane, the side surface Fh2 and the side surface Fh5 are parallel to the Y-Z plane, and the side surface Fh3 and the side surface Fh4 are parallel to the X-Z plane.

As illustrated in FIG. 8(a), the adjustment box Ci is a rectangular parallelepiped, and includes a bottom surface Fi1, side surfaces Fi2 to Fi5, and a top surface Fi6. In addition, as illustrated in FIG. 8(a), each surface Fi is parallel to one of the X-Y plane, the X-Z plane, and the Y-Z plane. Specifically, among the surfaces Fi1 to Fi6, the bottom surface Fi1 and the top surface Fi6 are parallel to the X-Y plane, the side surface Fi2 and the side surface F15 are parallel to the Y-Z plane, and the side surface Fi3 and the side surface Fi4 are parallel to the X-Z plane.

FIG. 8(b) is a diagram for explaining a configuration for generating the reference box Ch. A conceptual diagram of the reference box Ch as viewed from the Z-axis direction side (from above) is illustrated in a left side portion of FIG. 8(b). The reference box Ch of the present embodiment is generated so as to be able to store the head image Gsh. FIG. 8(b) illustrates the head image Gsh stored in the reference box Ch. As illustrated in FIG. 8(b), the side surface Fh2 of the reference box Ch passes through a point Ph2 at which the X coordinate is largest in the head image Gsh. The side surface Fh5 of the reference box Ch passes through a point Ph5 at which the X coordinate is smallest in the head image Gsh. The side surface Fh3 of the reference box Ch passes through a point Ph3 at which the Y coordinate is largest in the head image Gsh, and the side surface Fh4 of the reference box Ch passes through a point Ph4 at which the Y coordinate is smallest in the head image Gsh.

As understood from the above description, the side surfaces Fh2 to Fh5 of the reference box Ch are generated to surround four sides of the head image Gsh when viewed from the Z-axis direction side. In addition, a conceptual diagram of the head image Gsh viewed from the X-axis direction side is illustrated in a right side portion of FIG. 8(b). As illustrated in FIG. 8(b), the top surface Fh6 of the reference box Ch passes through a point Ph6 at which the Z coordinate is largest in the head image Gsh. As described above, the side surfaces Fh2 to Fh5 and the top surface Fh6 of the reference box Ch are generated to be in contact with the subject image Gh.

Meanwhile, in a case where the subject S is captured from above (from the Z-axis side), the lower portion of the subject S is not captured. From the above circumstances, the lower portion of the subject S is missing from the head image Gsh. In the present embodiment, in order to deform the shape of the head model Mh to approximate the shape of the subject S with high accuracy, it is necessary to generate the bottom surface Fh1 of the reference box Ch at a position corresponding to the lower end Pah of the head of the subject S.

However, since the lower portion of the subject S is not included in the head image Gsh, the point (lower end) at which the Z coordinate is smallest in the head image Gsh is different from the actual lower end (Pah) of the subject S. Therefore, if the bottom surface Fh1 of the reference box Ch is generated at a point where the Z coordinate is smallest in the head image Gsh, the shape of the head model Mh may not be deformed to approximate the shape of the subject S with high accuracy, In the above case, there is a disadvantage that the volume and the height of the subject S cannot be estimated with high accuracy.

In order to suppress the above inconvenience, the applicant has noticed that the lower end Pah of the subject S lying on the flat surface A is positioned on the flat surface A. In FIG. 8(b), a lower portion Sh of the head of the subject S, which is missing from the head image Gs, is indicated by a broken line. As described above, the reference plane Fa is generated at a position corresponding to the flat surface A. Therefore, even though the lower portion Sh of the subject S is not included in the head image Gs, it can be assumed that the point corresponding to the lower end Pah of the lower portion Sh in the three-dimensional image space is located on the reference plane Fa,

In consideration of the above circumstances, the present embodiment adopts a configuration in which the bottom surface Fh1 of the reference box Ch is located on the reference plane Fa, In other words, the above configuration is a configuration in which the volume of the subject S including the lower portion Sh that is missing in the subject image Gs is estimated using the subject image Gs on the assumption that the lower portion Sh of the subject S is in contact with the flat surface A at the lower end Pah.

FIG. 8(c) is a diagram for explaining a configuration for generating the adjustment box Ci. A conceptual diagram of the adjustment box Ci as viewed from the Z-axis direction side (from above) is illustrated in FIG. 8(c). The adjustment box Ci is generated so as to be able to store the head model Mh.

As illustrated in a right portion of FIG. 8(c), the top surface F16 of the adjustment box Ci passes through a point Qi6 at which the Z coordinate is largest in the head model Mh. As illustrated in a left portion of FIG. 8(c), the side surface Fi2 of the adjustment box Ci passes through a point Qi2 at which the X coordinate is largest in the head model Mh, and the side surface Fi5 of the adjustment box Ci passes through a point Qi5 at which the X coordinate is smallest in the head model Mh. The side surface Fi3 of the adjustment box Ci passes through a point Pi3 at which the Y coordinate is largest in the head model Mh, and the side surface Fi4 of the adjustment box Ci passes through a point Pi4 at which the Y coordinate is smallest in the head model Mh. The head model Mh represents the entire head including the lower portion. As illustrated in FIG. 8(c), the bottom surface Fi1 of the adjustment box Ci passes through a point Qi1 at which the Z coordinate is smallest in the head model Mh.

In the head model deformation process, the information processing device 10 deforms the head model Mh according to the size (shape) of the reference box Ch. Specifically, the information processing device 10 obtains a ratio Rhix (=Lhx/Lix) between a length Lhx of the reference box Ch in the X-axis direction illustrated in FIG. 8(b) and a length Lix of the adjustment box Ci in the X-axis direction illustrated in FIG. 8(c). In addition, the information processing device 10 obtains a ratio Rhiy (=Lhy/Liy) between a length Lhy of the reference box Ch in the Y-axis direction illustrated in FIG. 8(b) and a length Liy of the adjustment box Ci in the Y-axis direction illustrated in FIG. 8(c). Similarly, the information processing device 10 obtains a ratio Rhiz (=Lhz/Liz) between a length Lhz of the reference box Ch in the Z-axis direction illustrated in FIG. 8(b) and a length Liz of the adjustment box Ci in the Z-axis direction illustrated in FIG. 8(c).

After obtaining the ratio Rhix, the ratio Rhiy, and the ratio Rhiz (which may be collectively referred to as “ratios Rhi”), the information processing device 10 deforms (enlarges or reduces) the head model Mh using the ratios Rhi. Specifically, the information processing device 10 multiplies the head model Mh by Rhix in the X-axis direction, by Rhiy in the Y-axis direction, and by Rhiz in the Z-axis direction. According to the above head model deformation process, even when the lower portion of the subject S is not captured, the shape of the head model Mh is easily approximated to the shape of the head of the subject S with high accuracy by generating the reference box Ch on the assumption that the lower end Pah of the subject S is in contact with the flat surface A.

FIGS. 9(a) and 9(b) are diagrams for explaining the body model deformation process in detail. In the body model deformation process, similarly to the head model deformation process described above, the shape of the body model Mb is deformed using a reference box Cb and an adjustment box Cc.

Meanwhile, it is assumed in the present embodiment that the subject S is a neonate, and neonates often wear disposable diapers, In the above case, a waist image Gw in the body image Gb includes a large area representing a disposable diaper covering the waist of the neonate. In consideration of the above circumstances, the reference box Cb of the present embodiment is generated based on a chest image Gc in the body image Gb.

FIG. 9(a) is a diagram for explaining the reference box Cb, The reference box Cb is a rectangular parallelepiped, and is generated so as to be able to store the chest image Gc. Specifically, the reference box Cb includes a bottom surface Fb1, side surfaces Fb2 to Fb5, and an a top surface Fb6. Each of the above surfaces Fb1 to Fb6 is parallel to one of the X-Y plane, the X-Z plane, and the Y-Z plane as illustrated in FIG. 9(a). Specifically, among the surfaces Fb1 to Fb6, the bottom surface Fb1 and the top surface Fb6 are parallel to the X-Y plane, the side surface Fb2 and the side surface Fb5 are parallel to the Y-Z plane, and the side surface Fb3 and the side surface Fb4 are parallel to the X-Z plane.

As illustrated in FIG. 9(a), the top surface Fb6 is in contact with a point Pb6 at which the Z coordinate is largest in the chest image Gc. In addition, the side surface Fb2 is in contact with a point Pb2 at which the X coordinate is smallest in the chest image Gc, and the side surface Fb5 is in contact with a point Pb5 at which the X coordinate is largest in the chest image Gc. Similarly, the side surface Fb4 is in contact with a point Pb4 at which the Y coordinate is smallest in the chest image Gc, and the side surface Fb3 is in contact with a point Pb3 at which the Y coordinate is largest in the chest image Gc.

As described above, in a case where the subject S is captured from above (from the Z-axis side), the lower portion of the subject S is not captured. From the above circumstances, the lower portion of the subject S is missing from the chest image Go. In FIG. 9(a), a lower portion Sb of the chest of the subject S, which is missing from the chest image Gc, is indicated by a broken line.

Meanwhile, it is usual that, when the subject S lies on the flat surface A, a lower portion of the chest of the subject S is in contact with the flat surface A. In view of the above circumstances, in the present embodiment, the reference box Cb is generated on the assumption that the lower end Pac of the chest of the subject S is located on the flat surface A (similarly to the reference box Ch illustrated in FIG. 8(b) described above).

Specifically, as illustrated in FIG. 9(a), a lower portion Sc of the chest of the subject S is missing from the chest image Gc. In consideration of the above circumstances, in the present embodiment, instead of the lower end of the chest image Gc, the bottom surface Fb1 of the reference box Cb is generated on the reference plane Fa (flat surface A) where the actual lower end Pac of the chest is located. According to the above configuration, the weight or height of the subject S can be estimated with higher accuracy, for example, as compared with a configuration in which the bottom surface of the reference box Cb passes through the lower end of the chest image Gc.

FIG. 9(b) is a view for explaining the adjustment box Cc. The adjustment box Cc is a rectangular parallelepiped, and is generated so as to be able to store the chest model Mc. Specifically, the adjustment box Cc includes a bottom surface Fc1, side surfaces Fc2 to Fc5, and a top surface Fc6. Each of the above surfaces Fc1 to Fc6 is parallel to one of the X-Y plane, the X-Z plane, and the Y-Z plane as illustrated in FIG. 9(b). Specifically, among the surfaces Fc1 to Fc6, the top surface Fc6 and the bottom surface Fc1 are parallel to the X-Y plane, the side surface Fc2 and the side surface Fc5 are parallel to the Y-Z plane, and the side surface Fc3 and the side surface Fc4 are parallel to the X-Z plane.

As illustrated in FIG. 9(b), among the surfaces Fc1 to Fc6 of the adjustment box Cc, the top surface Fc6 is in contact with a point Qc6 at which the Z coordinate is largest in the chest model Mc. In addition, the side surface Fc2 is in contact with a point Qc2 at which the X coordinate is smallest in the chest model Mc, and the side surface Fc5 is in contact with a point Qc5 at which the X coordinate is largest in the chest model Mc. Similarly, the side surface Fc3 is in contact with a point Qc3 at which the Y coordinate is largest in the chest model Mc, and the side surface Fc4 is in contact with a point Qc4 at which the Y coordinate is smallest in the chest model Mc. The chest model Mc represents the entire shape of the chest including the lower portion. In consideration of the above circumstances, the bottom surface Fc1 of the adjustment box Cc passes through a lower end Qc1 at which the Z coordinate is smallest in the chest model Mc.

In the body model deformation process, the information processing device 10 deforms the chest model Mc according to the size (shape) of the reference box Cb. Specifically, the information processing device 10 obtains a ratio Rbox(=Lbx/Lcx) between a length Lbx of the reference box Cb in the X-axis direction illustrated in FIG. 9(a) and a length Lcx of the adjustment box Co in the X-axis direction illustrated in FIG. 9(b). In addition, the information processing device 10 obtains a ratio Rbcy (=Lby/Lcy) between a length Lby of the reference box Cb in the Y-axis direction illustrated in FIG. 9(a) and a length Lcy of the adjustment box Cc in the Y-axis direction illustrated in FIG. 9(b). Similarly, the information processing device 10 obtains a ratio Rbcz (=Lbz/Lcz) between a length Lbz of the reference box Cb in the Z-axis direction illustrated in FIG. 9(a) and a length Lcz of the adjustment box Cc in the Z-axis direction illustrated in FIG. 9(b).

After obtaining the ratio Rbcx, the ratio Rbcy, and the ratio Rbcz (which may be collectively referred to as “ratios Rbc”), the information processing device 10 deforms (enlarges or reduces) the body model Mb (chest model Mc+waist model Mw) based on the ratios Rbc. Specifically, the information processing device 10 multiplies the body model Mb by Rbcx in the X-axis direction, by Rbcy in the Y-axis direction, and by Rbcz in the Z-axis direction. According to the above body model deformation process, even when the lower portion of the subject S is not captured, the shape of the body model Mb can be deformed to approximate the shape of the body of the subject S with high accuracy by generating the reference box Cb on the assumption that the lower end Pac of the chest of the subject S is in contact with the flat surface A.

FIG. 10(a) is a diagram for explaining the volume estimation process. In the volume estimation process, a volume of the subject S is estimated. As described above, in the present embodiment, the head model Mh, the body model Mb, the arm model Ma, and the leg model Ml are deformed to approximate the shapes of the respective portions of the subject S by performing the head model deformation process (see FIGS. 8(a) to 8(c)), the body model deformation process (see FIGS. 9(a) and 9(b)), the arm model deformation process, and the leg model deformation process. The subject model M is generated by combining the above deformed models. FIG. 10(a) illustrates a simulated diagram of the subject model M.

In the volume estimation process, the information processing device 10 generates cross sections Mp illustrated in FIG. 10(a). Each cross section Mp is obtained by cutting the subject model M in parallel to the Y-Z plane while shifting the position in the X-axis direction. In FIG. 10(a), two adjacent ones of the cross sections Mp are extracted and shown. As illustrated in FIG. 10(a), the adjacent cross sections Mp are spaced apart from each other by a “distance Δx” in the X-axis direction. The distance Δx is sufficiently smaller than the size of the subject image Gs. When the distance Δx is sufficiently small, the volume V of the subject model M is calculated (approximated) according to the following Formula 2. Note that “An” in Formula 2 denotes an area of an n-th cross section Mp from the tip of the subject model M in the X-axis direction. In addition, “N” in Formula 2 denotes the total number of cross sections Mp.

$$V = {\sum }_{n = 1}^{n = N} {A}_{n} \times Δ x \qquad ( Mathematical formula 2 )$$

As described above, the shape (size) of the subject model M approximates the actual shape of the subject S. Therefore, the volume V of the subject model M calculated by the volume estimation process can be estimated as the actual volume of the subject S. In addition, the information processing device 10 calculates the weight W of the subject S by multiplying the volume V of the subject model M by the average density D.

FIG. 10(b) is a diagram for explaining a height estimation process. In the height estimation process, the height of the subject model M is calculated, and the calculation result is estimated as the height of the subject S. Specifically, in the height estimation process, the information processing device 10 detects predetermined feature points in the subject model M. In the height estimation process of the present embodiment, as illustrated in FIG. 10(b), a point Pa corresponding to the top of the head of the subject S, a point Pb corresponding to the shoulder joint of the subject S, a point Pc corresponding to the hip joint of the subject S, a point Pd corresponding to the knee joint of the subject S, and a point Pe corresponding to the ankle of the subject S are detected in the subject model M. Note that, as a technology for detecting feature points in the subject model M, for example, a technology described in Japanese Patent No. 6868875 can be adopted.

When detecting the feature points in the subject model M, the information processing device 10 calculates a horizontal distance Lab from the point Pa corresponding to the top of the head to the point Pb corresponding to the shoulder joint. In addition, the information processing device 10 calculates a horizontal distance Lbc from the point Pb corresponding to the shoulder joint to the point Pc corresponding to the hip joint, a distance Lcd from the point Pc corresponding to the hip joint to the point Pd corresponding to the knee joint, and a distance Ide from the point Pd corresponding to the knee joint to the point Pe corresponding to the ankle.

In addition, the information processing device 10 calculates the sum of the distance Lab, the distance Lbc, the distance Lcd, and the distance Ide as the height H of the subject model M. The height H of the subject model M is estimated as the height of the subject S represented by the subject model M. Note that the height estimation process can be changed as appropriate. For example, the feature points detected from the subject model M are not limited to the above example as long as the height of the subject model M can be measured.

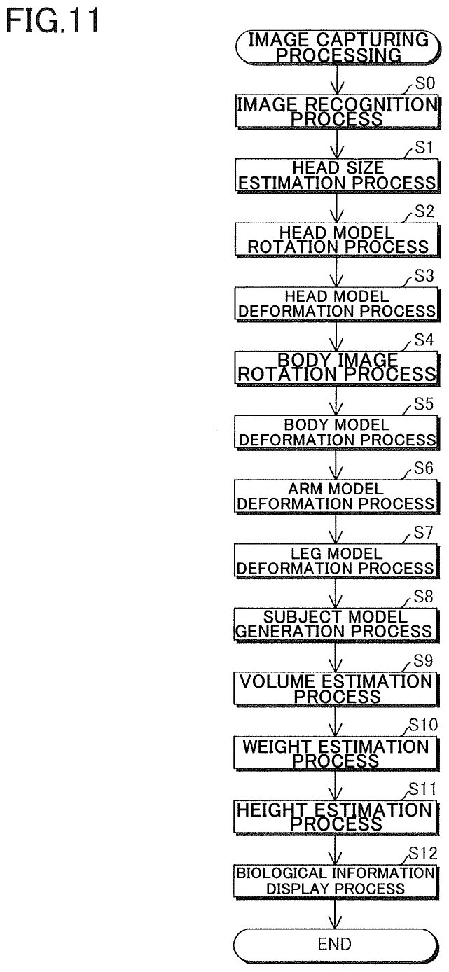

FIG. 11 is a flowchart of image capturing processing executed by the information processing device 10. As described above, the image capturing device 20 transmits image information Dg indicating a three-dimensional image G (including a subject image Gs) to the information processing device 10. The information processing device 10 executes the image capturing processing, for example, at the timing of acquiring (receiving) the image information Dg. However, the timing at which the image capturing processing is executed can be changed as appropriate.

When the image capturing processing is started, the information processing device 10 executes an image recognition process (S0). In the image recognition process, objects (including the subject S) shown in the three-dimensional image G indicated by the image information Dg are recognized. After executing the image recognition process, the information processing device 10 executes a head size estimation process (S1). In the head size estimation process, a rough shape (size) of the head model Mh is determined (see FIG. 7(a) described above). Furthermore, the information processing device 10 executes a head model rotation process after executing the head size estimation process. In the head model rotation process, the orientation of the head model Mh is rotated (adjusted) according to the orientation of the head image Gh (see FIG. 7(b) described above).

After executing the head model rotation process, the information processing device 10 executes a head model deformation process (S3), As described above, in the head model deformation process, the shape of the head model Mh is deformed to approximate the shape of the head of the subject S with high accuracy (see FIGS. 8(a) to 8(c) described above).

In addition, the information processing device 10 executes a body image rotation process (S4). In the body image rotation process, the body image Gsb is rotated so as to be parallel to the X-axis direction. Thereafter, the information processing device 10 executes a body model deformation process (S5). As described above, in the body model deformation process, the shape of the body model Mb is deformed to approximate the shape of the body of the subject S with high accuracy (see FIGS. 9(a) and 9(b) described above).

The information processing device 10 executes an arm model deformation process (S6) to deform the shape of the arm model Ma to approximate the shape of the arms of the subject S. In addition, the information processing device 10 executes a leg model deformation process (S7) to deform the shape of the leg model Ml to approximate the shape of the legs of the subject S.

Thereafter, the information processing device 10 executes a subject model generation process (S8). In the subject model generation process, a subject model M is generated by combining the head model Mh deformed in the most recent head model deformation process, the body model Mb deformed in the most recent body model deformation process, the arm model Ma deformed in the most recent arm model deformation process, and the leg model Ml deformed in the most recent leg model deformation process.

After executing the subject model generation process, the information processing device 10 executes a volume estimation process (S9). In the volume estimation process, a volume of a subject model M generated in the most recent subject model generation process is calculated (see FIG. 10(a) described above). After executing the volume estimation process, the information processing device 10 executes a weight estimation process (S10). In the weight estimation process, the information processing device 10 multiplies a volume of the subject model M calculated in the most recent volume estimation process by an average density, and stores a calculation result as the weight W of the subject S.

After executing the weight estimation process, the information processing device 10 executes a height estimation process (S11). In the height estimation process, a height of the subject model M generated in the most recent subject model generation process is measured, and a measurement result is stored as the height H of the subject S (see FIG. 10(b) described above). After executing the height estimation process, the information processing device 10 executes a biological information display process (S12). In the biological information display process, the weight W of the subject S calculated in the most recent weight estimation process and the height H of the subject S calculated in the most recent height estimation process are displayed on, for example, a monitor 104 of the incubator, When the biological information display process is executed, the information processing device 10 ends the image capturing processing.

### Second Embodiment

Other embodiments of the present invention will be described below. Note that, in each of the following exemplary embodiments, for elements of which actions or functions are equivalent to those in the first embodiment, the reference signs referred to in the description of the first embodiment will be used, and the detailed description thereof will be appropriately omitted.

Conventionally, a medical procedure in which a medical tube is inserted into a subject has been known. As the above medical procedure, for example, there is a medical procedure in which a gastric tube is inserted into a subject (a patient including a neonate) who is not able to orally ingest food. In the above medical procedure, the gastric tube is inserted through the nostril, passes through the esophagus, and reaches the inside of the stomach. In addition, in the above medical procedure, it is necessary to use a gastric tube having an appropriate length so that the tip (the exit of the tube) is located inside the stomach,

Meanwhile, the length from the nostril to the inside of the stomach varies depending on the subject. Thus, the appropriate length of the gastric tube varies from subject to subject. Therefore, conventionally, the sum of the length from the ear hole to the glabella and the length from the navel to the middle of the xiphoid process on the surface of the subject has been estimated as an appropriate length of the gastric tube.

However, it is necessary to touch the subject when measuring the length on the surface of the subject. In addition, depending on the subject, the above behavior may be considered invasive. In view of the above circumstances, an object of the second embodiment is to estimate an appropriate length of a medical tube (e.g., a gastric tube) to be inserted into a subject without touching the subject.

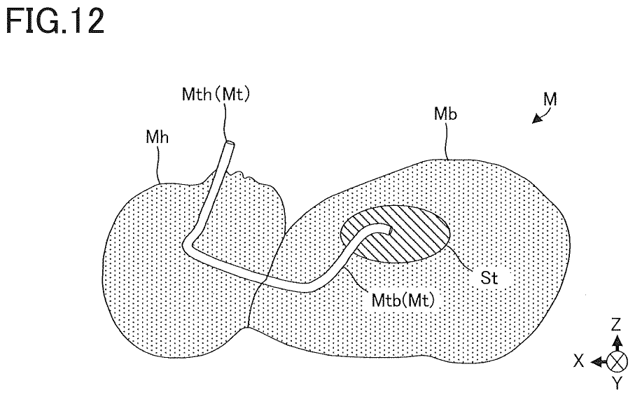

FIG. 12 is a conceptual diagram illustrating a head model Mh and a body model Mb of the second embodiment. In FIG. 12, the head model Mh and the body model Mb are shown in combination for the sake of explanation. FIG. 12 is a cross-sectional view illustrating the head model Mh and the body model Mb cut horizontally along the X-Z plane.

In the second embodiment, similarly to the first embodiment, a subject image Gs (three-dimensional image) representing a subject S is captured by the image capturing device 20. In addition, in the second embodiment, similarly to the first embodiment, the head model Mh, the body model Mb, the arm model Ma, and the leg model Ml are deformed based on the subject image Gs to generate a subject model M. In addition, in the second embodiment, similarly to the first embodiment, a volume of the subject model M is estimated as a volume of the subject S. In addition, a weight of the subject S is calculated from the estimated volume. In the second embodiment, similarly to the first embodiment, a height of the subject model M is estimated as a height of the subject S.

As illustrated in FIG. 12, the subject model M of the second embodiment includes a tube model Mt (Mth, Mtb). The tube model Mt represents a medical tube having an appropriate length to be inserted into the subject S indicated by the combination of the head model Mh and the body model Mb. Specifically, the tube model Mt indicates a shape of a gastric tube (an example of a medical tube) inserted into the subject S. The gastric tube inserted into the subject S bends in a path from the nostril to the stomach through the esophagus. The tube model Mt bends similarly to the gastric tube inserted into the subject S.

The tube model Mt is provided in a region corresponding to the medical tube inserted into the subject S in the combination of the head model Mh and the body model Mb. For example, the tube model Mt representing the gastric tube is located in a region corresponding to a path from the nostril to the inside of the stomach through the esophagus of the subject S. Note that a region St corresponding to the stomach of the subject S is shown in FIG. 12. As illustrated in FIG. 12, an end of the tube model Mt on the body model Mb side is located inside the region St.

As illustrated in FIG. 12, the tube model Mt includes a first tube model Mth provided inside the head model Mh and a second tube model Mtb provided inside the body model Mb, In the second embodiment, similarly to the first embodiment described above, the head model Mh is deformed to the size of the head of the subject S by executing a head model deformation process (see FIGS. 8(a) to 8(c)). In the head model deformation process, the first tube model Mth is deformed as an image integrated with the head model Mh. Furthermore, in the second embodiment, similarly to the first embodiment described above, the body model Mb is deformed to the size of the body of the subject S by executing a body model deformation process (see FIGS. 9(a) and 9(b)). In the body model deformation process, the second tube model Mtb is deformed as an image integrated with the body model Mb.

In the second embodiment described above, a subject model M that approximates the shape of the subject S is generated. In addition, the subject model M includes a tube model Mt (a combination of a first tube model Mth and a second tube model Mtb) deformed (expanded or contracted) to a length to be inserted into the subject S. Therefore, by calculating the length of the tube model Mt in the subject model M, a length of a medical tube to be inserted into the subject S can be grasped.

### Third Embodiment

In the first embodiment described above, a subject model M is generated, and a weight of the subject S is estimated by multiplying a volume of the subject model M by an average density. However, the configuration for estimating the weight of the subject S is not limited to the above example.

In the third embodiment, the subject model M is generated in the same manner as in the first embodiment described above. In the third embodiment, a length of a predetermined portion of the subject model M is specified as an explanatory variable X for estimating the weight of the subject S. The explanatory variable X is a length of a predetermined portion of the subject model M having a causal relationship with the weight of the subject S. In the third embodiment, n+1 (n is a positive integer) explanatory variables (X0, X1, X2, X3, . . . , and Xn) are specified. For example, a body length, a chest circumference, a chest width, a chest length, a foot length, a hand length, etc, of the subject model M are specified as the explanatory variables X.

After specifying the explanatory variables X, the information processing device 10 according to the third embodiment calculates (estimates) the weight W of the subject S by substituting the explanatory variables X into the following Formula 3. Note that coefficients k0 to kn in Formula 3 are determined by, for example, multiple regression analysis. However, the method of determining the coefficients k0 to kn is not limited to the multiple regression analysis.

W=K0×X0+k1×X1+k2××2+k3××3+ . . . +kn×Xn (Mathematical formula 3)

According to the third embodiment described above, effects similar to those of the first embodiment described above can be obtained. In the third embodiment, similarly to the second embodiment, the subject model M may include a tube model Mt. In addition, the configuration in which the weight W of the subject S is estimated by the explanatory variables (X0, X1, X2, X3, . . . , and Xn) is not limited to the above example. For example, a machine learning model machine-learned from teacher data including explanatory variables X and weights W may be used. Specifically, a machine learning model (a plurality of decision tree models) is generated using a random forest algorithm. In the above configuration, when a subject model M of the subject S is generated, explanatory variables X are specified from the subject model M, and when the explanatory variables X are input to the machine learning model, the weight W of the subject S is determined (estimated).

### <Modifications>

The above embodiments are variously modified. Specific aspects of modifications will be exemplified below. Any two or more aspects selected from the following examples can be combined as appropriate.

(1) In each of the above-described embodiments, the arm model deformation process can be appropriately changed. For example, as described above with reference to FIG. 6, the arm image Gsa includes a first arm image Gsa1 representing a portion from the shoulder to the elbow of subject S and a second arm image Gsa2 representing a portion from the elbow to the fingertip of subject S. In addition, as illustrated in FIG. 6, the arm model Ma includes a first arm model Ma1 representing a portion from the shoulder to the elbow of the subject S and a second arm model Ma2 representing a portion from the elbow to the fingertip of the subject S.

In the arm model generation process the information processing device 10 cylindrically approximates the first arm image Gsa1. Specifically, the information processing device 10 specifies the orientation of the first arm image Gsa1 (hereinafter, “orientation vector V”) by principal component analysis. The information processing device 10 generates a cylinder whose side surface substantially overlaps the first arm image Gsa1 (hereinafter, “cylinder Cg”), among cylinders whose center lines are parallel to the orientation vector V, by using the least squares method,

The information processing device 10 generates a cylinder that approximates the first arm model Ma1 (hereinafter, “cylinder Cm”) by a method similar to that used for generating the cylinder Cg from the first arm image Gsa1. The information processing device 10 calculates a ratio “Rg/Rm” obtained by dividing a diameter Rg (thickness) of a bottom surface of the cylinder Cg by a diameter Rm of a bottom surface of the cylinder Cm, and enlarges or reduces the thickness of the first arm model Ma1 according to the ratio “Rg/Rm”. In addition, the information processing device 10 calculates a ratio “Tg/Tm” obtained by dividing a height Tg (length) of the cylinder Cg by a height Tm of the cylinder Cm, and enlarges or reduces the length of the first arm model Ma1 according to the ratio “Tg/Tm”. According to the above configuration, the shape of the first arm model Ma1 is deformed to approximate the shape of the upper arm of the subject S. Similarly, the shape of the second arm model Ma2 is deformed using the second arm image Gsa2.

(2) In each of the above-described embodiments, the leg model deformation process can be appropriately changed. For example, as described above with reference to FIG. 6, the leg image Gsl includes a first leg image Gsl1 representing a portion from the base of the leg to the knee and a second leg image Gsl2 representing a portion from the knee to the toe. Each of the above images is distinguished using a segmentation technology. As illustrated in FIG. 6, the leg model Ml includes a first leg model Ml1 representing the thigh of the subject S and a second leg model Ml2 representing a portion from the knee to the toe of the subject S,

The information processing device 10 deforms the shape of the first leg model Ml1 using the first leg image Gsl1 by a method similar to the method of deforming the shape of the first arm model Ma1 in the above-described modification (1). According to the above configuration, the shape of the first leg model Ml1 is deformed to approximate the shape of the thighs of the subject S. In addition, the information processing device 10 deforms the shape of the second leg model Ml2 using the second leg image Gsl2 by a method similar to the method of deforming the shape of the first arm model Ma1. According to the above configuration, the shape of the second leg model Ml2 is deformed to approximate the shape of the leg from the knee to the toe of the subject S.

(3) In each of the above-described embodiments, the method of determining the reference plane Fa can be appropriately changed. For example, a configuration in which the positional relationship between the image capturing device 20 and the flat surface A is fixed is assumed. As the above configuration, it may be considered to fix the image capturing device 20 to the incubator. In the above configuration, the distance from the image capturing device 20 to the flat surface A is constant, and the reference plane Fa through which the flat surface image Ga passes is common regardless of the three-dimensional image G. In the above configuration, the reference plane Fa can be stored in advance in the information processing device 10. Therefore, there is an advantage that the process for specifying the reference plane Fa from the flat surface image Ga can be omitted.

(4) In the first embodiment described above, in the head size estimation process, the shape (size) of the head of the subject S is estimated by spherically approximating the head image Gsh. Instead of the above configuration, a configuration in which the head image Gsh is elliptically approximated may be adopted.

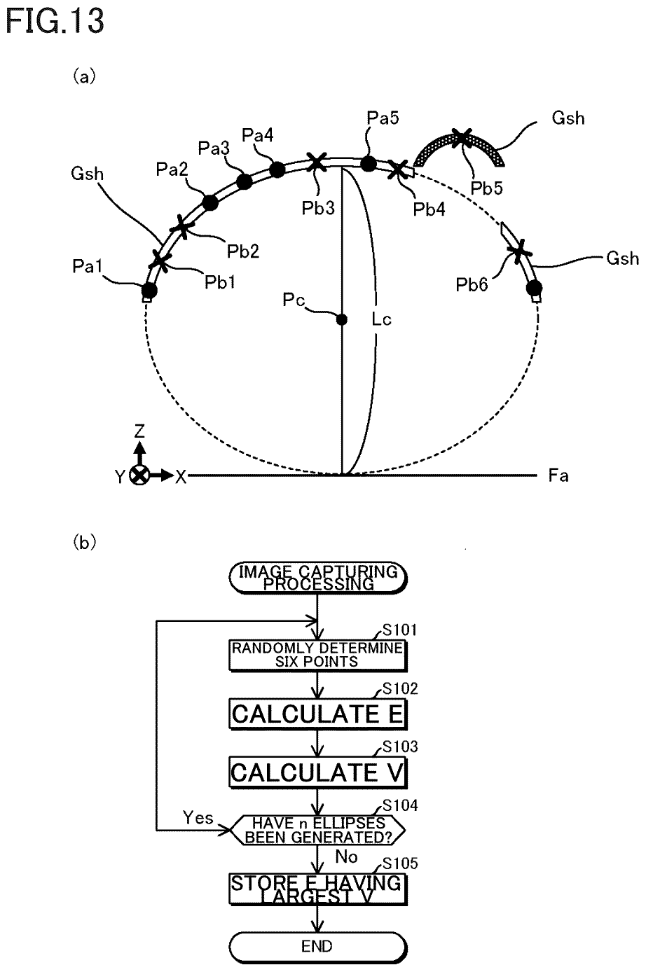

FIGS. 13(a) and 13(b) are diagrams for explaining a modification in which the head image Gsh can be elliptically approximated. In the modification, the head image Gsh is cut in parallel to the Z-X plane and at equal intervals in the Y-axis direction, and the cross sections are elliptically approximated. In addition, the model Mh is deformed (enlarged or reduced) to the size of the image generated by superimposing the approximated ellipses. FIG. 13(a) illustrates the head image Gsh before being elliptically approximated. As described above, the reference plane Fa (flat surface A) side of the head of the subject S is not captured and is therefore missing from the head image Gsh. In FIG. 13(a), the shape of the head of the subject S missing from the head image Gsh is indicated by a broken line.

Meanwhile, the subject S (neonate) may touch his/her own head with his/her hand. In addition, a medical tube may be attached to the subject S inside the incubator. In the above case, a partial portion of the head is shielded by the hand (arm) of the subject S or the tube, and the shielded portion is missing from the head image Gsh. In the specific example of FIG. 13(a), it is assumed that a partial portion of the head is shielded by the arm of the subject S. In the above case, as illustrated in FIG. 13(a), the head image Gsh on the lower side (the side opposite to the Z-axis direction) of the arm image Gsa is missing. In addition, the arm image Gsa can be recognized as a partial portion of the head image Gsh.

If an image including the arm image Gsa and the head image Gsh is elliptically approximated, there is a high possibility that the approximated ellipse does not match the actual shape of the head of the subject S. Therefore, in the above case, the actual shape of the head of the subject S is not estimated with high accuracy. In consideration of the above circumstances, the modification has a configuration in which the above inconvenience is suppressed. The above configuration will be described in detail below.

The ellipse is expressed by “Xᵢ²+AXᵢYᵢ+BYᵢ²+CXᵢ+DYᵢ+E” (hereinafter, “equation Eᵢ”) using five unknowns A to E. Therefore, by substituting a combination of coordinates (Xᵢ, Yᵢ) of five or more points in the head image Gsh into the equation Et, an ellipse passing through the head image Gsh is obtained (five coefficients A to E, which are unknowns, are obtained). In the modification, the equation Eᵢ is obtained using coordinates (Xᵢ, Yᵢ) of six points in the head image Gsh. In addition, in the modification, equations Eᵢ of n ellipses (for example, n=1000) are obtained while randomly changing the combinations of points in the head image Gsh.

In addition, after the equation Eᵢ of the ellipse is obtained, the number of points on the head image Gsh located on the ellipse (hereinafter, “evaluation value Vᵢ”) is counted. In the above modification, it can be estimated that an ellipse having a larger evaluation value Vᵢ is more approximate to the actual shape of the head of the subject S. For example, in the specific example of FIG. 13(a), it is assumed that an equation Eᵢ of an ellipse is obtained by a combination of points Pa1 to Pa6 in the head image Gsh, and an equation Eᵢ of another ellipse is obtained by a combination of points Pb1 to Pb6. In addition, in the specific example of FIG. 13(a), it is assumed that all the points Pa1 to Pa6 are located in the head image Gsh, while the point Pb5 among the points Pb1 to Pb6 is located in the arm image Gsa rather than the head image Gsh. In the above case, the ellipse obtained by the combination of the points Pa1 to Pa6 is likely to approximate the actual shape of the head of the subject S. That is, the ellipse obtained by the combination of the points Pa1 to Pa6 has a large area that overlaps the head image Gsh, and it is likely that the evaluation value Vᵢ is large. On the other hand, the ellipse obtained by the combination of the points Pb1 to Pb6 is unlikely to approximate the actual shape of the head of the subject S. That is, the ellipse obtained by the combination of the points Pb1 to Pb6 has a small area that overlaps the head image Gsh, and it is likely that the evaluation value Vᵢ is small.

As understood from the above description, an ellipse having a larger evaluation value Vᵢ is more approximate to the actual shape of the head of the subject S. In consideration of the above circumstances, in the modification, the evaluation values Vᵢ of the n ellipses are obtained, and the ellipse having the largest evaluation value Vᵢ is estimated as an ellipse representing the actual shape of the head of the subject S.

FIG. 13(b) is a flowchart of elliptical approximation processing. An equation Eᵢ of an ellipse that approximates the shape of the head of the subject S is determined by elliptical approximation processing. Specifically, when the elliptical approximation processing is started, the information processing device 10 randomly determines six points P in the subject image Gs (S101), and calculates an equation Eᵢ of an ellipse passing through the points P (S102). Furthermore, the information processing device 10 obtains the number of points on a subject image Gs; located on the ellipse of the equation Eᵢ as an evaluation value Vᵢ (S103). Thereafter, the information processing device 10 determines whether evaluation values Vᵢ of n ellipses have been obtained (S104). When the number of ellipses for which the evaluation values Vᵢ have been obtained is smaller than n (S104: No), the information processing device 10 repeatedly executes the above-described steps S101 to S104. On the other hand, when the number of ellipses for which the evaluation values Vᵢ have been obtained has reached n (S104: Yes), the information processing device 10 stores an equation Eᵢ of an ellipse having the largest evaluation value Vᵢ among the n ellipses (S105), and ends the elliptical approximation processing.

Note that the length of the radius of the ellipse obtained by the elliptical approximation processing should be half the length from the reference plane Fa to the vertex of the subject image Gs (Lc in FIG. 13(a)). Therefore, a difference (error) between the length of the radius of the ellipse obtained by the elliptical approximation processing and the length Lc may be obtained, and it may be determined that there is an error when the difference exceeds a predetermined threshold. When it is determined that there is an error, the elliptical approximation processing may be executed again. In addition, an inclination of the ellipse may be calculated from the equation Et, and the orientation of the ellipse may be adjusted according to the calculation result.

(5) In each of the above-described embodiments, the information about the subject S to be estimated is not limited to the weight and the height. For example, the information processing device 10 (measurement unit 16) may be configured to estimate a head circumference, a chest circumference, an abdominal circumference, an arm thickness, and a leg thickness of the subject S.

Specifically, the information processing device 10 may be configured to measure a head circumference, a chest circumference, an abdominal circumference, an arm thickness, and a leg thickness of the generated subject model M, and estimate respective measurement results as the head circumference, the chest circumference, the abdominal circumference, the arm thickness, and the leg thickness of the subject S.

<Summary of Actions and Effects of Examples of Aspects of

### Present Embodiment>

### <First Aspect>

An information processing device (10) according to the present aspect includes: an acquisition unit (11) configured to acquire image information (Dg) indicating a three-dimensional image (G) obtained by capturing a subject(S) lying on a flat surface (A) from above; a recognition unit (12) configured to recognize a subject image (Gs) representing the subject among objects displayed in the three-dimensional image; and an estimation unit (13) configured to estimate a shape of the subject including a lower portion missing in the subject image using the subject image, assuming that the lower portion of the subject is in contact with the flat surface, in which physical information (volume, weight, height, etc.) of the subject is calculated using the estimated shape of the subject. According to the present aspect, the physical information of the subject can be estimated without touching the subject. Furthermore, according to the aspect, the physical information of the subject can be estimated with high accuracy even from the subject image in which the lower portion of the subject is missing. Note that, in the present invention, the wording “estimating the shape of the subject” means that it is sufficient as long as the physical information of the subject S can be calculated, and is not limited to estimating the shape of the subject S in detail.

### <Second Aspect>

The information processing device according to the present aspect further includes a storage unit (14) configured to store model information indicating a shape of a model (such as a head model) of the subject, in which the recognition unit is configured to recognize a flat surface image (Ga) representing a flat surface (A) in the three-dimensional image, and the estimation unit is configured to: specify a reference plane (Fa) passing through the flat surface image; deform the model according to a ratio between a distance (Lhz, Lbz) in a vertical axis direction (Z-axis direction) from a subject point (Ph6 in FIG. 8(b) and Pb6 in FIG. 9(a)) in the subject image to the reference plane and a distance (Liz, Lcz) in the vertical axis direction from a corresponding point (Qi6 in FIG. 8(c) and Qc6 in FIG. 9(b)) in the model corresponding to the subject point to a lower end of the model; and estimate a shape of the deformed model as the shape of the subject. According to the present aspect, the physical information of the subject can be estimated with high accuracy, for example, as compared with a configuration in which the physical information of the subject is estimated using only the subject image in which the lower portion is missing.

### <Third Aspect>

In the information processing device according to the present aspect, a weight of the subject is calculated as the physical information, According to the present aspect, the weight of the subject can be estimated without touching the subject.

### <Fourth Aspect>

In the information processing device according to the present aspect, a height of the subject is calculated as the physical information. According to the present aspect, the height of the subject can be estimated without touching the subject.

### <Fifth Aspect>

In the information processing device (10) according to the present aspect, the model includes a tube model (Mt) representing a medical tube (e.g., a gastric tube) inserted into the subject, and the estimation unit is configured to deform the model (head model Mh and chest model Mb) other than the tube model integrally with the tube model. According to the present aspect, the optimal length of the medical tube can be estimated without touching the subject.

### <Sixth Aspect>

An information processing device (10) according to the present aspect includes: an acquisition unit (11) configured to acquire image information (Dg) indicating a three-dimensional image (G) obtained by capturing a subject (S) lying on a flat surface (A) from above; a recognition unit (12) configured to recognize a subject image (Gs) representing the subject and a flat surface image (Ga) representing the flat surface among images representing respective objects displayed in the three-dimensional image; a storage unit (14) configured to store model information indicating a shape of a model of the subject; and an estimation unit (13) configured to estimate a weight of the subject, in which the estimation unit is configured to: specify a reference plane passing through the flat surface image; deform the model according to a ratio between a distance in a vertical axis direction from a subject point in the subject image to the reference plane and a distance in the vertical axis direction from a corresponding point in the model corresponding to the subject point to a lower end of the model; specify a length of a predetermined portion in the deformed model as an explanatory variable for estimating a weight of the subject; and estimate the weight of the subject using the specified explanatory variable. According to the present aspect described above, effects similar to those of the third aspect described above can be obtained.

### <Seventh Aspect>

The neonate weight estimation method according to the present aspect includes: capturing a three-dimensional image of a neonate; estimating, by a computer, a shape of the neonate from the three-dimensional image (S1 to S9 in FIG. 11); and estimating, by the computer, a weight of the neonate from the estimated shape (S10 in FIG. 11). Touching a neonate to measure physical information with a scale may be an invasive action. According to the present aspect, the weight of the neonate is estimated without touching the neonate, which is advantageous in that the number of invasive actions can be reduced.

### EXPLANATION OF THE REFERENCE NUMERALS

10 Information processing device, 11 Acquisition unit, 12 Recognition unit, 13 Estimation unit, 14 Storage unit, 15 Calculation unit, 16 Measurement unit, 20 Image capturing device

1. An information processing device comprising:
an acquisition unit configured to acquire image information indicating a three-dimensional image obtained by capturing a subject lying on a flat surface from above;
a recognition unit configured to recognize some images as a subject image representing the subject and to recognize other images as a flat surface image representing the flat surface among images included in the three-dimensional image; and
an estimation unit configured to:
calculate, based on a position of the flat surface image, a reference plane passing through the flat surface image and passing through a position corresponding to the flat surface that is shielded by the subject and is missing from the flat surface image,
and to estimate a shape of the subject including a lower portion missing in the subject image using the subject image, using a distance in a vertical axis direction from the reference plane corresponding to the flat surface to a subject point in the subject image,
wherein physical information of the subject is calculated using the estimated shape of the subject, and
wherein both the subject image and the flat surface image used in estimating the shape of the subject are obtained from one said three-dimensional image.

2. The information processing device according to claim 1, further comprising:
a storage unit configured to store model information indicating a shape of a model of the subject, wherein
the estimation unit is configured to
deform the model according to a ratio between a distance in a vertical axis direction from a subject point in the subject image to the reference plane and a distance in the vertical axis direction from a corresponding point in the model corresponding to the subject point to a lower end of the model, and
estimate a shape of the deformed model as the shape of the subject.

3. The information processing device according to claim 1, wherein a weight of the subject is calculated as the physical information.

4. The information processing device according to claim 1, wherein a height of the subject is calculated as the physical information.

5. The information processing device according to claim 2, wherein
the model includes a tube model representing a medical tube inserted into the subject, and
the estimation unit is configured to enlarge the tube model in conjunction with enlarging the model other than the tube model, as well as can reduce the tube model in conjunction with reducing the model other than the tube model
wherein a length of the medical tube necessary for the subject can be estimated from a length of the deformed tube model.

6. The information processing device according to claim 2, wherein the processing device
specifies a length of a predetermined portion in the deformed model as an explanatory variable for estimating a weight of the subject, and
estimates the weight of the subject using the specified explanatory variable.

7. A neonate physical information estimation method comprising:
capturing a three-dimensional image of a neonate lying on a flat surface;
recognizing some images as a subject image representing the subject and to recognize other images as a flat surface image representing the flat surface among images included in the three-dimensional image;
calculating, by a computer, based on a position of the flat surface image, a reference plane passing through the flat surface image and passing through a position corresponding to the flat surface that is shielded by the subject and is missing from the flat surface image;
estimating, by the computer, a shape of the neonate including a lower portion missing in the subject image, using a distance in a vertical axis direction from a reference plane corresponding to the flat surface to a subject point in the subject image; and
estimating, by the computer, physical information of the neonate from the estimated shape, wherein both the subject image and the flat surface image used in estimating the shape of the neonate are obtained from one said three-dimensional image.

8. The information processing device according to claim 2, wherein a weight of the subject is calculated as the physical information.

9. The information processing device according to claim 2, wherein a height of the subject is calculated as the physical information.
